(19) United States
(12) Patent Application Publication
(10) Pub. No.: US 2025/0353747 A1
(43) Pub. Date: Nov. 20, 2025

(54) EFFECTIVE CALCIUM PHOSPHATE RADIATIVE COOLING MATERIALS

(71) Applicants: Northeastern University, Boston, MA (US); Planck Energies Inc., Brighton, MA (US)

(72) Inventors: Andrew Caratenuto, Medford, MA (US); Yi Zheng, Canton, MA (US); Nathaniel LeCompte, Brighton, MA (US)

(21) Appl. No.: 19/208,825

(22) Filed: May 15, 2025

Related U.S. Application Data
(60) Provisional application No. 63/647,849, filed on May 15, 2024.

(51) Int. Cl. C01B 25/32; A61K 8/24; A61Q 17/04; C01B 25/42; C04B 14/36; C04B 103/54; C04B 111/00; C08K 3/32; C09D 5/33; C09D 7/40; C09D 7/61

(52) U.S. Cl. CPC C01B 25/32; A61K 8/24; A61Q 17/04; C01B 25/42; C04B 14/366; C09D 5/004; C09D 7/61; C09D 7/67; C09D 7/68; C09D 7/69; C01P 2002/02; C01P 2002/82; C01P 2004/03; C01P 2004/50; C01P 2004/61; C01P 2004/62; C01P 2004/64; C01P 2006/60; C04B 2103/54; C04B 2111/00448; C08K 2003/325; C08K 2201/005; C08K 2201/011

(57) ABSTRACT

Disclosed herein are highly solar-reflective passive radiative cooling materials in powder form based on calcium phosphates. These materials can be synthesized at a low material cost approaching that of paint additives available on the market, and they are safe, non-toxic, and environmentally-friendly. These materials achieve solar reflectance values ranging from 93% to nearly 98%, making them extremely effective passive radiative cooling materials.

### RELATED APPLICATION

This application claims the benefit of priority to U.S. Provisional Application No. 63/647,849, filed May 15, 2024.

### GOVERNMENT SUPPORT

This invention was made with government support under Grant No. 1941743 and 2321446 awarded by the National Science Foundation. The government has certain rights in the invention.

### BACKGROUND

Passive radiative cooling materials can cool structures without requiring any electricity due largely to their high solar reflectance values. However, achieving materials with near-perfect solar reflectance is difficult in practice, and often mandates complex material structures, expensive fabrication methods, and/or environmentally harmful constituents. There is a need to develop low-cost, environmentally friendly and highly effective passive radiative cooling materials in order to reduce GHG emissions and heat-related illness and death.

Calcium phosphates (CaPs) represent an important and diverse family of biomaterials. Their compositional similarity to bone minerals found within the human body, as well as their osteoconductivity and biodegradability, enable important opportunities in dental procedures, drug delivery, and other vital biomedical practices. As such, the synthesis, characterization, and application of calcium phosphates have continually garnered research attention, especially in recent decades. Simultaneously, calcium phosphates have also been leveraged within green technologies, such as within sustainable fertilizers and passive radiative cooling materials.

The specific constituency of calcium phosphate compounds is largely dependent upon the synthesis method, which can have pronounced effects on properties and target application. The wet chemical precipitation method is one of the most commonly studied methods. This is due to the convenience of being performed at ambient pressure, and the fact that synthesis parameters such as reaction temperature and pH may be easily modified to tune the resultant calcium phosphates. Many previous works focus on a narrow range of synthesis parameters, including only several pH values or temperatures. In addition, optical properties, as are relevant to potential applications as passive radiative cooling materials, are not typically presented.

### SUMMARY OF THE INVENTION

In certain aspects, the present disclosure provides materials, comprising one or more calcium phosphates, wherein the material is a polydisperse powder comprising a plurality of particles of differing sizes.

In further aspects, described herein are processes of preparing a solar reflective material, comprising:
i) combining a first solution comprising a calcium-containing reagent with a second solution comprising a phosphate-containing reagent at a reaction temperature and a reaction pH, thereby producing a calcium-phosphate mixture comprising calcium-phosphate;
ii) allowing the calcium phosphate to precipitate from the calcium-phosphate mixture; and
iii) calcining the calcium phosphate, thereby producing the solar reflective material.

### BRIEF DESCRIPTION OF THE DRAWINGS

FIG. 1 shows FTIR transmittance spectra of CaPs synthesized at reaction temperatures of (a) 20° C. and (b) 60° C., with commercial HA for comparison.

FIG. 2 shows side-by-side comparison of FTIR transmittance spectra for controlled pH CaPs at different synthesis temperatures.

FIG. 3 shows (a) side-by-side comparison of FTIR transmittance spectra for natural pH CaPs at different reaction temperatures. FTIR transmittance spectra of (b) CaP-Nat-20 and (c) CaP-Nat-60 before and after calcination, showing brushite as the precursor material. (d) Starting solution and time-dependent reaction pHs during CaP-Nat-60 synthesis. (e) Post-calcination mass yield of selected CaPs synthesized at 60° C. Dotted lines represent the theoretical maximum yields of product constituent compounds.

FIG. 4 shows SEM images of pH-controlled CaP samples at various reaction temperatures, at low and high magnifications. Arrows highlight larger, more amorphous agglomerates.

FIG. 5 shows SEM images of (a)-(c) CaP-Nat-20, (d)-(e) CaP-Nat-60, and (g)-(i) commercial HA.

FIG. 6 shows spectral reflectance in the solar wavelength region for CaPs at reaction temperatures of (a) 20° cand (b) 60° C., with commercial HA shown for reference. (c) Average reflectance of CaPs normalized with respect to the solar radiation spectrum. The commercial HA (not shown) has a normalized value of 0.848%±0.004. CaP-Nat samples were characterized after dispersing in ethanol and allowing the resulting film to dry, because the morphology of the CaP-Nat samples made forming a bulk puck from the sample difficult (as was done for other samples). The normalized reflectance for CaP-Nat samples in the bulk puck form is estimated by measuring the difference between these sample preparation methods for other CaP samples. (d) Solar radiation spectrum. (d) Refractive indices of constituent CaP materials. Backscattering coefficients of CaP materials as functions of incident wavelength and spherical particle size, (f) with no weighting and (g) weighted with respect to the solar radiation spectrum. (h) Ratio of backscattering coefficients calculated at a refractive index of 1.64 to those calculated at a refractive index of 1.59, exemplifying the proportional increase in scattering due to a greater refractive index.

### DETAILED DESCRIPTION OF THE INVENTION

The present invention is based on the surprising discovery of solar reflective calcium phosphate materials. The morphological and compositional properties of these materials can be tailored by varying the parameters of a wet chemical synthesis method. Such materials and syntheses are discussed herein.

In certain aspects, the present disclosure provides materials, comprising one or more calcium phosphates, wherein the material is a polydisperse powder comprising a plurality of particles of differing sizes.

In certain embodiments, the one or more calcium phosphates are selected from calcium pyrophosphate (CPP), biphasic calcium phosphate (BCP), hydroxyapatite (HA), calcium-deficient HA (CDHA), tricalcium phosphate (TCP), and a combination of any of them.

In certain embodiments, the one or more calcium phosphates is calcium pyrophosphate. In certain embodiments, the calcium pyrophosphate is α-calcium pyrophosphate (α-CPP). In certain embodiments, the calcium pyrophosphate is β-calcium pyrophosphate (β-CPP). In certain embodiments, the calcium pyrophosphate is γ-calcium pyrophosphate (γ-CPP).

In certain embodiments, the one or more calcium phosphates is tricalcium phosphate (TCP). In certain such embodiments, the tricalcium phosphate is α-tricalcium phosphate (α-TCP). In certain embodiments, the tricalcium phosphate is β-tricalcium phosphate (β-TCP).

In certain embodiments, the one or more calcium phosphates comprise hydroxyapatite and calcium-deficient hydroxyapatite.

In certain embodiments, the plurality of particles has a distribution of particle sizes of about 1 nm to about 400 nm. In certain embodiments, the plurality of particles has a distribution of particle sizes of about 20 nm to about 200 nm. In certain embodiments, the plurality of particles has a distribution of particle sizes of about 20 nm to about 40 nm. In certain embodiments, the plurality of particles has a distribution of particle sizes of about 40 nm to about 60 nm. In certain embodiments, the plurality of particles has a distribution of particle sizes of about 60 nm to about 80 nm. In certain embodiments, the plurality of particles has a distribution of particle sizes of about 80 nm to about 100 nm. In certain embodiments, the plurality of particles has a distribution of particle sizes of about 100 nm to about 120 nm. In certain embodiments, the plurality of particles has a distribution of particle sizes of about 120 nm to about 140 nm. In certain embodiments, the plurality of particles has a distribution of particle sizes of about 140 nm to about 160 nm. In certain embodiments, the plurality of particles has a distribution of particle sizes of about 160 nm to about 180 nm. In certain embodiments, the plurality of particles has a distribution of particle sizes of about 180 nm to about 200 nm.

In certain embodiments, the plurality of particles form a plurality of aggregates. In certain embodiments, the plurality of aggregates comprises amorphous aggregates. In certain embodiments, the plurality of aggregates comprises microstructures or nanostructures or both. In certain such embodiments, the plurality of aggregates comprises microstructures and nanostructures.

In certain embodiments, the plurality of aggregates comprises one or more morphologies selected form shard-like, plate-like, particles, rods, spherules, and a combination of any of them. In certain embodiments, the plurality of aggregates comprises at least two different morphologies.

In certain embodiments, the plurality of aggregates has a distribution of aggregate sizes of about 200 nm to about 10 μm. In certain embodiments, the plurality of aggregates has a distribution of aggregate sizes of about 800 nm to about 2 μm. In certain embodiments, the plurality of aggregates has a distribution of aggregate sizes of about 800 nm to about 900 nm. In certain embodiments, the plurality of aggregates has a distribution of aggregate sizes of about 900 nm to about 1 μm. In certain embodiments, the plurality of aggregates has a distribution of aggregate sizes of about 1 μm to about 1.1 μm. In certain embodiments, the plurality of aggregates has a distribution of aggregate sizes of about 1.1 μm to about 1.2 μm. In certain embodiments, the plurality of aggregates has a distribution of aggregate sizes of about 1.2 μm to about 1.3 μm. In certain embodiments, the plurality of aggregates has a distribution of aggregate sizes of about 1.4 μm to about 1.5 μm. In certain embodiments, the plurality of aggregates has a distribution of aggregate sizes of about 1.5 μm to about 1.6 μm. In certain embodiments, the plurality of aggregates has a distribution of aggregate sizes of about 1.6 μm to about 1.7 μm. In certain embodiments, the plurality of aggregates has a distribution of aggregate sizes of about 1.7 μm to about 1.8 μm. In certain embodiments, the plurality of aggregates has a distribution of aggregate sizes of about 1.9 μm to about 2 μm.

In certain embodiments, the material has a refractive index of about 1.55 to about 1.68. In certain embodiments, the material has a refractive index of about 1.58 to about 1.65. In certain embodiments, the material has a refractive index of about 1.59. In certain embodiments, the material has a refractive index of about 1.60. In certain embodiments, the material has a refractive index of about 1.61. In certain embodiments, the material has a refractive index of about 1.62. In certain embodiments, the material has a refractive index of about 1.63. In certain embodiments, the material has a refractive index of about 1.64. In certain embodiments, the material has a refractive index of about 1.65.

In certain embodiments, the material has normalized spectral reflectance values of about 0.85 to about 0.98 in the solar radiation spectrum (250-2500 nm). In certain embodiments, the material has normalized spectral reflectance values of about 0.85 in the solar radiation spectrum (250-2500 nm). In certain embodiments, the material has normalized spectral reflectance values of about 0.86 in the solar radiation spectrum (250-2500 nm). In certain embodiments, the material has normalized spectral reflectance values of about 0.87 in the solar radiation spectrum (250-2500 nm). In certain embodiments, the material has normalized spectral reflectance values of about 0.88 in the solar radiation spectrum (250-2500 nm). In certain embodiments, the material has normalized spectral reflectance values of about 0.89 in the solar radiation spectrum (250-2500 nm). In certain embodiments, the material has normalized spectral reflectance values of about 0.90 in the solar radiation spectrum (250-2500 nm). In certain embodiments, the material has normalized spectral reflectance values of about 0.90 to about 0.98 in the solar radiation spectrum (250-2500 nm). In certain embodiments, the material has normalized spectral reflectance values of about 0.90 to about 0.91 in the solar radiation spectrum (250-2500 nm). In certain embodiments, the material has normalized spectral reflectance values of about 0.91 to about 0.92 in the solar radiation spectrum (250-2500 nm). In certain embodiments, the material has normalized spectral reflectance values of about 0.92 to about 0.93 in the solar radiation spectrum (250-2500 nm). In certain embodiments, the material has normalized spectral reflectance values of about 0.93 to about 0.98 in the solar radiation spectrum (250-2500 nm). In certain embodiments, the material has normalized spectral reflectance values of about 0.93 to about 0.94 in the solar radiation spectrum (250-2500 nm). In certain embodiments, the material has normalized spectral reflectance values of about 0.94 to about 0.95 in the solar radiation spectrum (250-2500 nm). In certain embodiments, the material has normalized spectral reflectance values of about 0.95 to about 0.96 in the solar radiation spectrum (250-2500 nm). In certain embodiments, the material has normalized spectral reflectance values of about 0.96 to about 0.97 in the solar radiation spectrum (250-2500 nm). In certain embodiments, the material has normalized spectral reflectance values of about 0.97 to about 0.98 in the solar radiation spectrum (250-2500 nm).

In yet further aspects, the present invention describes additives for a passive radiative cooling paint or coating, comprising the materials described herein.

In still further aspects, the present invention describes paints, comprising the materials described herein.

In certain aspects, the present invention describes building materials, comprising the materials described herein. In certain embodiments, the building material is selected from shingles, wood, and concrete.

In further aspects, the present invention describes sunscreens, comprising the materials described herein.

In yet further aspects, the present invention describes pigments, comprising the materials described herein.

In certain embodiments, the material reflects sunlight.

In still further aspects, the present invention describes processes of preparing a solar reflective material, comprising:
i) combining a first solution comprising a calcium-containing reagent with a second solution comprising a phosphate-containing reagent at a reaction temperature and a reaction pH, thereby producing a calcium-phosphate mixture comprising calcium-phosphate;
ii) allowing the calcium phosphate to precipitate from the calcium-phosphate mixture; and
iii) calcining the calcium phosphate, thereby producing the solar reflective material.

In certain embodiments, the molar ratio of the calcium-containing reagent to the phosphate-containing reagent is about 0.50 to about 1.50. In certain embodiments, the molar ratio of the calcium-containing reagent to the phosphate-containing reagent is about 0.70 to about 1.00. In certain embodiments, the molar ratio of the calcium-containing reagent to the phosphate-containing reagent is about 0.70 to about 0.75. In certain embodiments, the molar ratio of the calcium-containing reagent to the phosphate-containing reagent is about 0.72. In certain embodiments, the molar ratio of the calcium-containing reagent to the phosphate-containing reagent is about 0.75 to about 0.80. In certain embodiments, the molar ratio of the calcium-containing reagent to the phosphate-containing reagent is about 0.80 to about 0.85. In certain embodiments, the molar ratio of the calcium-containing reagent to the phosphate-containing reagent is about 0.85 to about 0.90. In certain embodiments, the molar ratio of the calcium-containing reagent to the phosphate-containing reagent is about 0.90 to about 0.95. In certain embodiments, the molar ratio of the calcium-containing reagent to the phosphate-containing reagent is about 0.95 to about 1.00. In certain embodiments, the molar ratio of the calcium-containing reagent to the phosphate-containing reagent is about 1.00.

In certain embodiments, the process is performed at ambient pressure.

In certain embodiments, the process further comprises stirring the calcium-phosphate mixture at ambient temperature prior to step ii). In certain such embodiments, stirring the calcium-phosphate mixture occurs for about 90 minutes. In certain embodiments, stirring the calcium-phosphate mixture occurs without applying heat.

In certain embodiments, allowing the calcium-phosphate to precipitate from the calcium-phosphate mixture comprises allowing the calcium-phosphate mixture to rest for about 24 hours.

In certain embodiments, provided herein, the process further comprises filtering the calcium-phosphate.

In certain embodiments, the process further comprises drying the calcium-phosphate at a drying temperature for a drying duration. In certain embodiments, the drying temperature is about 50° C. to about 100° C. In certain embodiments, the drying temperature is about 50° C. to about 60° C. In certain such embodiments, the drying temperature is about 60° C. In certain embodiments, the drying temperature is about 60° C. to about 70° C. In certain embodiments, the drying temperature is about 70° C. to about 80° C. In certain embodiments, the drying temperature is about 80° C. to about 90° C. In certain embodiments, the drying temperature is about 90° C. to about 100° C.

In certain embodiments, the drying duration is about 24 hours to about 96 hours. In certain embodiments, the drying duration is about 24 hours to about 48 hours. In certain embodiments, the drying duration is about 24 hours. In certain embodiments, the drying duration is about 48 hours to about 72 hours. In certain embodiments, the drying duration is about 72 hours to about 96 hours.

In certain embodiments, the process further comprises grinding the calcium phosphate.

In certain embodiments, the calcium-containing reagent is calcium nitrate. In certain embodiments, the calcium-containing reagent is calcium nitrate tetrahydrate.

In certain embodiments, the phosphate-containing reagent is ammonium phosphate dibasic.

In certain embodiments, the reaction temperature is about 20° C. to about 60° C. In certain embodiments, the reaction temperature is about 20° C. to about 30° C. In certain embodiments, the reaction temperature is about 30° C. to about 40° C. In certain embodiments, the reaction temperature is about 40° C. to about 50° C. In certain embodiments, the reaction temperature is about 50° C. to about 60° C. In certain embodiments, the reaction temperature is about 20° C. or about 60° C. In certain embodiments, the reaction temperature is about 20° C. In certain embodiments, the reaction temperature is about 60° C.

In certain embodiments, the reaction pH is uncontrolled. In certain embodiments, the reaction pH is controlled.

In certain embodiments, the pH is controlled by adding ammonium hydroxide.

In certain embodiments, the controlled reaction pH is about 4 to about 11. In certain embodiments, the controlled reaction pH is about 8 to about 11. In certain embodiments, the controlled reaction pH is about 8. In certain embodiments, the controlled reaction pH is about 9. In certain embodiments, the controlled reaction pH is about 10. In certain embodiments, the controlled reaction pH is about 11.

In certain embodiments, calcining the calcium-phosphate occurs at a calcination temperature; and the calcination temperature is about 700° C.

In certain embodiments, calcining the calcium-phosphate occurs for a calcination duration; and the calcination duration is about 1 hour.

In certain embodiments, the reaction temperature is about 20° C.; and the reaction pH is uncontrolled. In certain embodiments, the reaction temperature is about 20° C.; and the reaction pH is about 8. In certain embodiments, the reaction temperature is about 20° C.; and the reaction pH is about 9. In certain embodiments, the reaction temperature is about 20° C.; and the reaction pH is about 10. In certain embodiments, the reaction temperature is about 20° C.; and the reaction pH is about 11.

In certain embodiments, the reaction temperature is about 60° C.; and the reaction pH is uncontrolled. In certain embodiments, the reaction temperature is about 60° C.; and the reaction pH is about 8. In certain embodiments, the reaction temperature is about 60° C.; and the reaction pH is about 9. In certain embodiments, the reaction temperature is about 60° C.; and the reaction pH is about 10. In certain embodiments, the reaction temperature is about 60° C.; and the reaction pH is about 11.

In certain embodiments, the process is performed on a liter-scale.

Disclosed herein are highly solar-reflective passive radiative cooling materials in powder form based on hydroxyapatite (HAP) and closely related calcium phosphate biomaterials. These materials can be synthesized at a low material cost approaching that of paint additives available on the market. As biomaterials, they are safe, non-toxic, and environmentally-friendly. Most importantly, the various forms of these materials achieve solar reflectance values ranging from over 90% to nearly 98%, making them extremely effective passive radiative cooling materials.

The materials disclosed herein are produced in sizes and particle sizes that are perfectly suited for scattering broadband solar radiation.

Additionally, HAP and other calcium phosphates are not currently used as reflective pigments in existing industry. Thus, HAP represents a new material for use in a solar reflective application.

While modifications in the temperature and/or pH of the synthesis process do produce slightly different constituencies of calcium-phosphate compounds, the high solar reflectance is largely insensitive to these conditions.

The materials described herein provide ultra-high solar reflectance from a non-toxic, eco-friendly biomaterial. Additionally, the materials are low-cost. The materials also provide very high UV reflectance, which is a known drawback of the industry-standard reflective pigment Titanium Dioxide (TiO₂).

Materials described herein may be useful in the following applications:
Additive for passive radiative cooling paints and coatings
Additive for existing building materials, such as shingles, wood, and concrete
UV blocking agent for sunscreen
White pigment for extruded polymer components

### Experimental

### Procedure for Material Preparation:

Add 24.48 g Ca(NO₃)₂ (calcium nitrate tetrahydrate) to 400 ml DI water
Separately, add 15.84 g (NH₄)₂HPO₄ (Ammonium phosphate dibasic) to 400 ml DI water
Stir each for 30 min while bringing up to reaction temperature
   Reaction temperatures tested: ambient (no heating), 60° C.
Slowly add ammonium hydrogen phosphate solution to calcium nitrate solution under slow magnetic stirring, about 5 ml at a time (continue heating if applicable)
   pH Variation: To control pH, dibasic ammonium hydroxide is used to raise the pH of the aqueous reagent solutions (Ca and PO4 solutions) to the intended pH (i.e. 9, 10, etc.) prior to the reaction.
Stir product at ambient (no heat applied) for 90 minutes
Remove from stirring and let rest covered overnight
Vacuum filter product as-is using Grade 4 filter paper
After filtration, place on a sheet of foil and dry filter paper+product in drying oven overnight
Remove product from filter paper and grind to powder using a mechanical grinder
Calcine at 700° C. for 1 h (1 h ramp up from ambient temperature, 1 h at 700° C., 1 h ramp down to ambient temperature)

Described herein are detailed characterizations of calcium phosphates synthesized using a wet chemical precipitation method across a wide range of pH (approximately 4 to 11) and reaction temperature (20° C. to 60° C.) values. Fourier transform infrared (FTIR) spectroscopy and scanning electron microscopy (SEM) may offer valuable conclusions on the impact of synthesis parameters on chemical constituency, particle size, and micro and nanomorphology.

Furthermore, optical characterizations of broadband solar reflectance are provided to assess the viability of various calcium phosphates as passive radiative cooling materials. These spectra may elucidate further chemical behaviors and resonances not typically reported for calcium phosphates.

Described herein is a wet chemical precipitation method for calcium phosphate synthesis was explored at a large range of reactant pH and reaction temperature values. The composition of the resulting calcium phosphates were dispersed between many different phases, including hydroxyapatite, tricalcium phosphate, and calcium pyrophosphate. Both compositional and morphological differences were explored as a result of these differing synthesis parameters. Within alkaline solutions, reactant pH was found to have only minor differences on composition as characterized by FTIR. Higher synthesis temperature was generally noted to yield less amorphous structures and smaller feature sizes, though several outliers manifested. The most severe differences in composition resulted from uncontrolled pH values, which produced a vastly different phase of calcium phosphates using the same reactant proportions due to the acidic reaction environment. In addition, all synthesized calcium phosphates were shown to exhibit extremely high broadband solar reflectance. Scattering trends of calcium phosphate particle dispersions were estimated, which may indicate that the greatest contributor to their high solar reflectance performance was their broad distribution of feature sizes that favor scattering in the solar wavelength region. While slight differences in refractive index between different calcium phosphates emerged as a factor, morphology and feature size may affect reflectance trends. In sum, the capability of this wet chemical precipitation method to produce highly reflective CaP powders—along with the relative insensitivity of resultant reflectance to synthesis parameters and composition—may offer a promising and scalable material for use as a filler or pigment in paint formulations. Furthermore, the use of biomaterials such as CaPs in paints and coatings may significantly promote sustainability, eco-friendliness, and non-toxicity. This factor may prove valuable to an industry which is under consistent pressure to reduce its environmental impact.

### Definitions

As used herein, the term “calcining” refers to a process of heating a solid material to drive off approximately 90 weight percent or more of volatile chemically bound components (e.g., organic components).

### EXAMPLES

### Example 1: Synthesis of Calcium Phosphates

### Materials

Calcium nitrate tetrahydrate (CAS 13477-34-4), ammonium phosphate dibasic (CAS 7783-28-0), ammonium hydroxide (CAS 1336-21-6), and commercial hydroxyapatite (CAS 1306-06-5, 2.5 μm particle size) were purchased from Sigma-Aldrich.

### Synthesis

Synthesis was carried out based partially on the procedures of Wang et al, but instead with a reactant Ca/P ratio of 0.72. In an exemplary synthesis, 24.48 g calcium nitrate tetrahydrate (Ca(NO3)2) were added to 400 ml DI water. Separately, 15.84 g ammonium phosphate dibasic ((NH4)2HPO4) were added to 400 ml DI water. Both beakers were stirred for 30 min while bringing them to the reaction temperature. Reaction temperatures were either ambient (approximately 20° C.) or 60° C. In the case of controlled pH, ammonium hydroxide was slowly added to each solution until each reactant solution reaches the desired pH. No ammonium hydroxide was added for the uncontrolled (or “natural”) pH. Next, the ammonium hydrogen phosphate solution was slowly added to the calcium nitrate solution under slow magnetic stirring, about 5 ml at a time, while keeping the solution at the reaction temperature. The addition rate was approximately 25 ml min−1. Afterwards, the mixed product was stirred with no heating for 90 min, followed by resting overnight. The mixture precipitated, and the white calcium phosphate powder was seen to separate at the bottom of the beaker. Next, the product was filtered using Grade 4 filter paper under vacuum filtration and rinsed with DI water. The filtered calcium phosphate was placed in a drying oven at 60° C. overnight, followed by grinding using a mechanical grinder. Finally, the ground powder was calcined in an oven at 700° C. for 1 h.

### Example 2: Characterization of Calcium Phosphates

FTIR transmissivity spectra were obtained using a Jasco FTIR 6600. The KBr pellet method was used to prepare samples for this measurement. For the SEM characterizations, the Supra 25 SEM was used with an acceleration voltage of 5 kV. A 10 nm layer of a gold/palladium mixture was coated on all samples prior to SEM characterizations.

Reflectance spectra in the solar wavelength region (200-2500 nm) was obtained using a Jasco V770 spectrometer equipped with an ISN-923 integrating sphere at an angle of 6°. A Spectralon SRS-99-010 diffuse reflectance standard, along with associated absolute reflectance calibration data for the standard, was used to calibrate the V770 spectrometer to measure the absolute reflectance of samples. As the sample window of the integrating sphere is oriented vertically, the samples must be formed into a freestanding bulk form for characterization. Thus, for solar reflectance characterizations, calcium phosphate samples were pressed into a small puck using a cylindrical mold. This method preserves the surface roughness at the interface and is a good representation of the solar reflectance of the bulk material considering surface scattering effects. Specific samples, such as the natural pH calcium phosphates, are not possible to press into a puck using this method due to their surface characteristics. To remedy this, CaP-Nat samples were dispersed in ethanol and deposited in a 3 mm layer atop a wood substrate. After drying, they were placed within the spectrometer for testing in the same orientation as bulk puck samples. Evaluation of control samples using both puck and deposition methods indicated that the puck method—a more accurate representation of the bulk material—yields normalized reflectance values 3.4-4.6% higher than the deposition method. This may be attributed to to a higher proportion of surface adsorption which may occur during the deposition process. For this reason, normalized reflectance data for CaP-Nat samples was presented in the deposition form and the estimated puck form. This strategy provided a rough estimate of the true bulk reflectance of the CaP-Nat samples considering all scattering effects, despite the characterization limitation inherent from the particle sizes and material properties of these samples.

### Results

### FTIR Characterization

Various calcium phosphate (CaP) samples were synthesized using a wet chemical precipitation method, using a notably low Ca/P ratio of 0.72. Each sample has a prescribed pH (uncontrolled, or 8-11) and reaction temperature (20-60° C.). For brevity, samples were abbreviated based on synthesis parameters (e.g., “CaP-8-60” represents the calcium phosphate sample with a reaction pH and temperature of 8 and 60° C., respectively). Samples of natural pH were denoted with the abbreviation “Nat” in place of the pH (e.g., “CaP-Nat-60”).

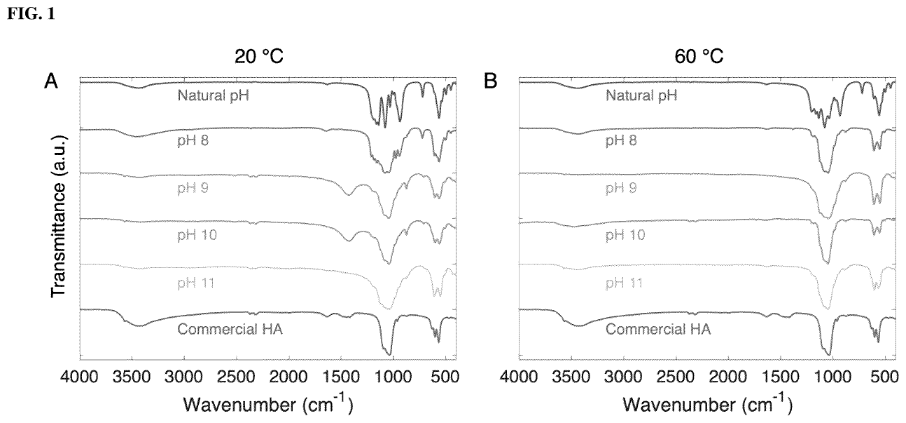

First, FTIR spectra of all samples, as well as a commercial hydroxyapatite (HA), were compared to ascertain the specific composition of the CaPs, as shown in FIG. 1. The HA shows the peaks expected from phosphate resonances (471, 567, 603, 962, 1036 and 1094 cm⁻¹) and hydroxyl resonances (632 and 3571 cm⁻¹). This sample also showed indications of water (broad peaks around 1630 and 3430 cm⁻¹) and carbonate (873 and 1400-1460 cm⁻¹), both of which are common for HA due to the adsorption of atmospheric H₂O and CO2, respectively. No other notable peaks were seen, indicating that this sample may be a good representation of stoichiometric HA.

### Controlled pH

In CaP samples with a pH of 8 and above (with the exception of CaP-8-20), HA peaks were visible but less prominent. These samples show very obvious phosphate resonances, concentrated in the 500-600 cm⁻¹ and 900-1100 cm⁻¹ regions. However, the hydroxyl resonances at 632 and 3571 cm⁻¹ are less obvious in these samples as compared to pure HA. New resonances also appear in these samples—e.g., around 431, 880, 1122, 1185, and 1208 cm⁻¹. Some phosphate resonances typical of HA remain, but many either shift slightly (for example, 567 and 603 cm⁻¹ respectively shift lower and higher) or become intermingled with new nearby resonances (as is seen in the 950-1100 cm⁻¹ region). These observations may indicate two important qualities of these samples. Firstly, the reduction of the hydroxyl bands, shifts in phosphate resonances, and several new resonances (especially those in the 1100-1200 cm⁻¹ region) may be indicative of biphasic calcium phosphate (BCP)—a mixture of β-tricalcium phosphate (β-TCP) and stoichiometric HA. The wet chemical synthesis procedures used herein are known to yield calcium-deficient HA (CDHA)—non-stoichiometric HA with calcium or hydroxyl vacancies—through the precipitation of amorphous calcium phosphate followed by transformation to CDHA. The composition of the resulting CDHA has been observed to be highly pH dependent and somewhat reaction temperature dependent, with higher pH and reaction temperature resulting in a higher Ca/P ratio tending towards stoichiometric HA. At high calcination temperatures, CDHA may form TCP, with the specific phase of TCP being dependent on calcination temperature. j-TCP may be obtained via calcination of CDHA at or above 750° C., and temperatures around 900° C. are typically employed. However, conversion of CDHA to β-TCP at temperatures as low as 650° C. has been reported, and may be attributed to the nanomorphology of the CDHA. Hence, the employed synthesis procedure is capable of producing BCP, as is observed from the FTIR characterization. At calcination temperatures above those used here (e.g., 750-900° C.), a more complete transition of CDHA to β-TCP is expected for these cases when CDHA is present, while α-TCP may be obtained by calcining above 1400° C.

The second notable quality is the presence of unreacted CDHA which remains in the samples after calcination. As 700-800° C. is noted as the lower bound for the conversion of CDHA to β-TCP, some CDHA may survive the calcination process (especially given the relatively short calcination time of 1 h). Similar mixtures of CDHA and β-TCP have been reported previously at a similar calcination temperature range employed herein, noting that at slightly higher temperatures, all CDHA may disappear. FTIR spectra show evidence of this: the resonances present around 880, 1186, and 1207 cm⁻¹ correspond to the hydrogen phosphate (HPO4) group of CDHA. It is important to note that peaks around 875-880 cm⁻¹ may also indicate carbonate groups from adsorbed CO2, as previously noted. However, adsorbed carbonate will also show peaks around 1400-1460 cm⁻¹, while HPO4 will not. Hence, the 873 cm⁻¹ resonance of the commercial HA may be due to carbonate, while the peaks near 875-880 cm⁻¹ in many CaP samples may be attributed instead to HPO4 groups of un-reacted CDHA. This is further supported by the FTIR curves of CaP-9-20 and CaP-10-20. These curves may show evidence of carbonate adsorption in the 1400-1460 cm⁻¹ region, in addition to evidence of hydrogen phosphates close to 1200 cm⁻¹. As a consequence, the 875-880 cm⁻¹ resonance is much stronger than in other samples, which may be attributed to the combination of both carbonate and hydrogen phosphate resonances at this location increasing the prominence of this peak. These samples show greater carbonate adsorption, which may be due to minor differences in sample storage time or location.

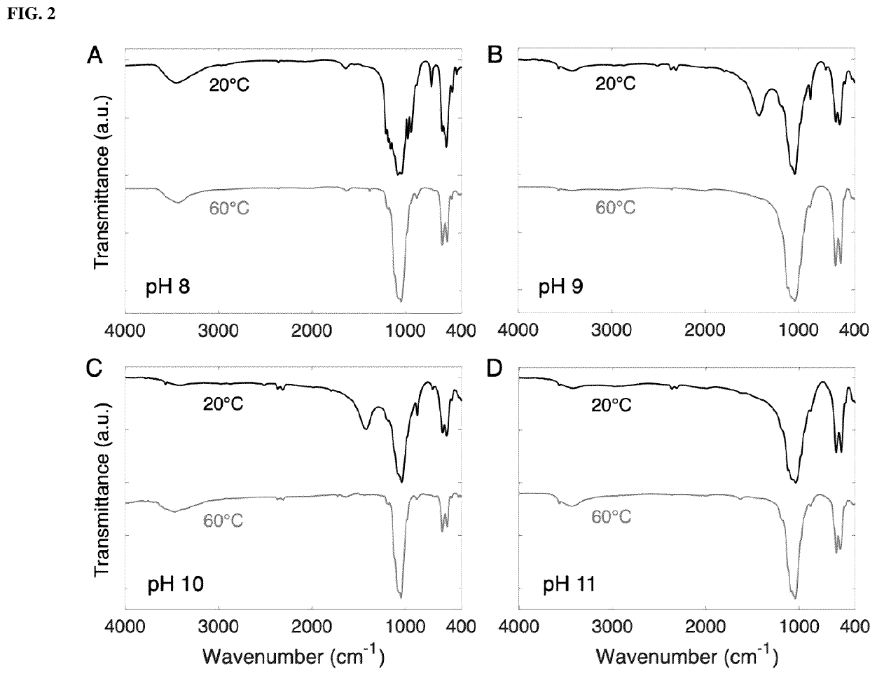

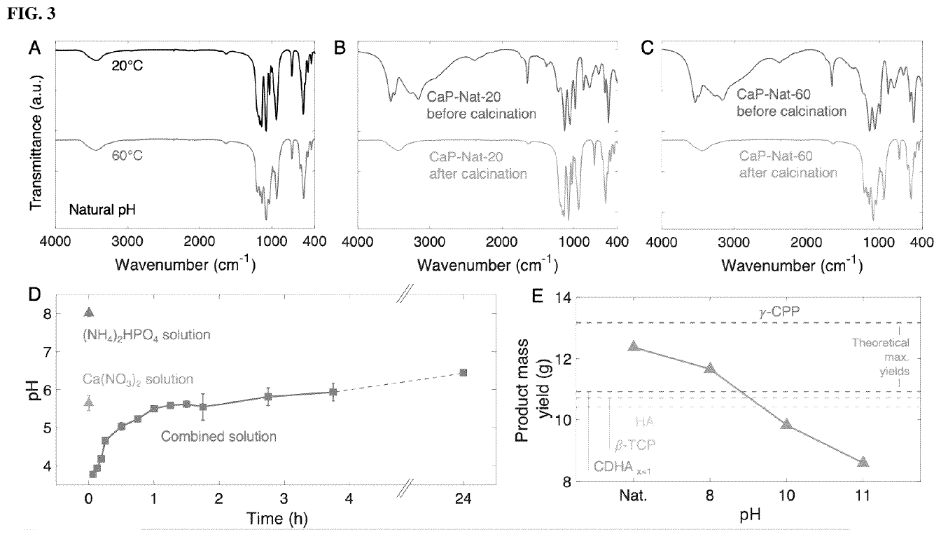

As mentioned previously, pH and reaction temperature may be driving factors in the product Ca/P ratio of the wet chemical precipitation process, with higher pH and reaction temperatures tending towards stoichiometric HA. Besides CaP-8-20, samples with a pH of 8 and above show slightly more prominent hydroxyl peaks as pH is increased. The re-action temperature for samples of pH 9 and above may play a more minor role in the resulting FTIR spectra, as seen in FIGS. 2 and 3. However, the complex composition of HA, CDHA, and β-TCP that is yielded by these procedures complicates a more detailed assignment of composition. The overlapping nature of the phosphate peaks of HA and CDHA makes it difficult to quantitatively estimate the relative proportions of these compounds, especially when β-TCP is also present in the mixture. However, the prominence of hydrogen phosphate peaks in all samples, together with the diminished indications of the hydroxyl group, may suggest that both β-TCP and CDHA dominate with respect to stoichiometric HA. Finally, based upon available literature, increasing reaction temperature above the range studied here may further enhance Ca/P ratios towards stoichiometric HA.

### Natural (Uncontrolled) pH

Far more variation was seen in samples once the pH 8 threshold is approached. Both CaP samples at natural pH have far more resonances than other samples. Many new peaks appeared, and others shifted or disappeared, with respect to higher pH samples. Upon comparison with previous literature, the FTIR spectra of both CaP-Nat-20 and CaP-Nat-60 may indicate a near total composition of γ-calcium pyrophosphate (γ-CPP). Such a disparate composition in comparison to samples of elevated pH may be explained by the different outcome of the wet chemical synthesis at lower pH.

Without pH modification, the initial pHs of calcium and phosphate source solutions prior to combination are roughly to 6 and 8, respectively (FIG. 3d). When combined, the reaction pH of the mixed solutions may be reduced due to the formation of ammonium nitrate, a byproduct of the precipitation mechanism. Due to the lower starting pH of the reactants in the natural case, reaction pH may dip far lower than in controlled pH syntheses. The mixture pH is transient in nature as the reaction progresses, but was found to vary from just below 4 to approximately 6 during the first several hours of the CaP-Nat-60 reaction (FIG. 3). In weakly acidic environments (pH 4-6), the wet chemical synthesis process outlined herein will produce dicalcium phosphate dihydrate (also known as brushite) instead of CDHA. After precipitating for 24 h, the pH stabilizes at about 6.5. Hence, the synthesis procedure described herein executed without pH control may provide an environment for brushite formation, as has been exemplified previously. In contrast to CDHA, which is thermally stable up to approximately 700-800° C., brushite may react to form CPP at calcination temperatures as low as 200° C., with a complete reaction of brushite being noted as low as 500° C. Thus, the prescribed synthesis procedure with natural pH may produce CPP from brushite. The calcination temperature of 700° C. may result in a complete transformation of brushite to γ-CPP; at higher temperatures (in the range of 800-1000° C.), β-CPP may be expected, and at 1200° C. or above, α-CPP may be produced. This is further supported by the pre-calcination FTIR spectra of CaP-Nat-20 and CaP-Nat-60 (FIG. 3), both of which show the expected resonance bands in the phosphate region as well as the distinct four-peak OH stretching vibration of brushite. It should be noted that if the pH were maintained below the natural case and/or with a higher reaction temperature, different polymorphs of calcium phosphate hydrates may result.

The CaP-8-20 sample shows an interesting intermediate case of resultant CaP composition. The starting pH of both reactants is 8, but as previously noted, will fall below 8 during the reaction when the solutions are combined due to ammonium nitrate byproducts. At or below this pH value, this same wet chemical synthesis process is capable of producing dicalcium phosphates in addition to CDHA. After calcination at the proper temperature(s), this may yield a combination of γ-CPP and β-TCP. As noted previously, higher reaction temperatures may play a similar role as the pH in supporting greater product Ca/P ratios. This may provide an explanation for the stark difference in FTIR spectra of CaP-8-20 and CaP-8-60: while the combination of reaction temperature and pH in CaP-8-60 appears sufficient to keep the product Ca/P ratio at or above 1.5 (which may eliminate the potential for CPP to form after calcination), the reaction conditions of CaP-8-20 may result in a product Ca/P ratio below 1.5. This may produce both brushite and CDHA during precipitation and may result in a mixture of γ-CPP and R-TCP after calcination. There is no indication of hydroxyl or hydrogen phosphate groups in CaP-8-20 after calcination, which may indicate that the proportions of un-reacted CDHA and/or stoichiometric HA are minimal in this case. This may be attributed to both the reduced quantity of CDHA in the precipitate as well as the lowered Ca/P ratio expected from this synthesis procedure, which may yield j-TCP than stoichiometric HA after calcination.

A notable aspect of all synthesized samples is their capability to form CaPs with higher product Ca/P ratios than those of the input reactants. The reactant Ca/P ratio is quite low—only 0.72—and CPP, TCP, and HA have product Ca/P ratios of 1.0, 1.5, and 1.67, respectively. Yet, FTIR evidence of CPP, TCP, and HA were found in all samples, with higher pH values and reaction temperatures appearing to promote higher Ca/P ratio products, as noted in the literature. This behavior was previously noted and investigated by Zyman et al., who hypothesized that higher product ratios are obtained due to the thermal decomposition of calcium-based compounds during the calcination process. Mass yield data of selected CaPs (FIG. 3e) may provide further evidence that the reaction pH has the largest impact on product constituency. CaPs synthesized at natural pH have a significantly higher mass yield than those with raised pH. This may be because the theoretical maximum mass yield of γ-CPP (based on the input calcium source mass) is 20-25% higher than that of β-TCP, CDHA, and HA. Samples synthesized at 60° C. which show FTIR evidence of R-TCP, CDHA, and HA (i.e., higher pH samples) have mass yields below the theoretical thresholds of these compounds, supporting previously noted FTIR trends. In addition, CaP-Nat-60 and CaP-8-60 have mass yields above these thresholds but below the threshold of γ-CPP. This matches with the obvious FTIR signatures of γ-CPP seen in these two samples. Another apparent trend is the reduction in product yield as the synthesis pH is increased. This may indicate that the synthesis pH impacts resultant CaP constituency and may produce higher Ca/P ratio compounds at higher pHs even when a notably sub-stoichiometric input is employed. As a result, product mass may be reduced as this Ca deficiency becomes more pronounced. Finally, while specific phase or Ca/P ratios were not identified for these compounds (as is often performed via XRD), it is emphasized that the literature notes agreement in both phase identification and trends between FTIR and XRD. In addition, as the literature contains many deep studies on these parameters, and the emphasis of the study described herein is on optical properties, a general phase and trend identification using FTIR is deemed sufficient.

### Microstructure Investigation

Scanning electron microscopy (SEM) images provide details of the morphology of the CaP samples. Despite the vast range of synthesis conditions explored, as well as the disparity in CaP composition of the products between samples, many morphological characteristics of these samples are quite similar. As will be described, this may be reasonable based on the choice of synthesis parameters and comparisons to literature.

Many conditions which control CaP morphology using similar synthesis methods have been proposed in the literature. For example, higher calcination temperatures may provide larger particle sizes, reaction pH and temperature may have highly diverse impacts on resulting morphology, and solvent choice, reactant addition rate, or the addition of Schiff bases may also influence morphological shape and scale. Despite this, there may be similarities in the micro- and nanostructural characteristics of CaPs, even with differing compositions such as HA/CDHA, β-TCP, and γ-CPP. A high level of agglomeration into micron-scale superstructures may be quite common, especially when dispersants are not employed. These aggregate structures may take the form of macroparticles, as well as shard-like or plate-like geometries. The primary CaP particles which comprise these structures may be sub-micron, with particle radii generally spanning from 20 to several hundred nanometers.

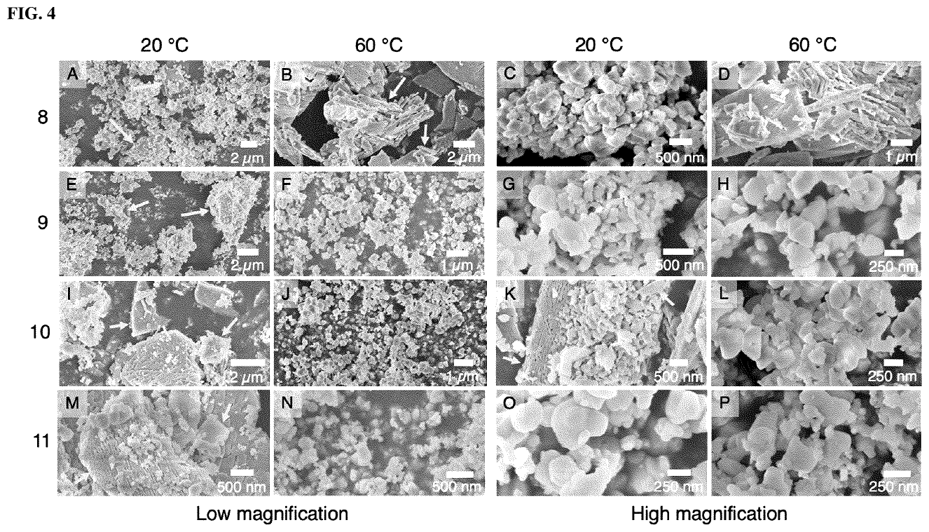

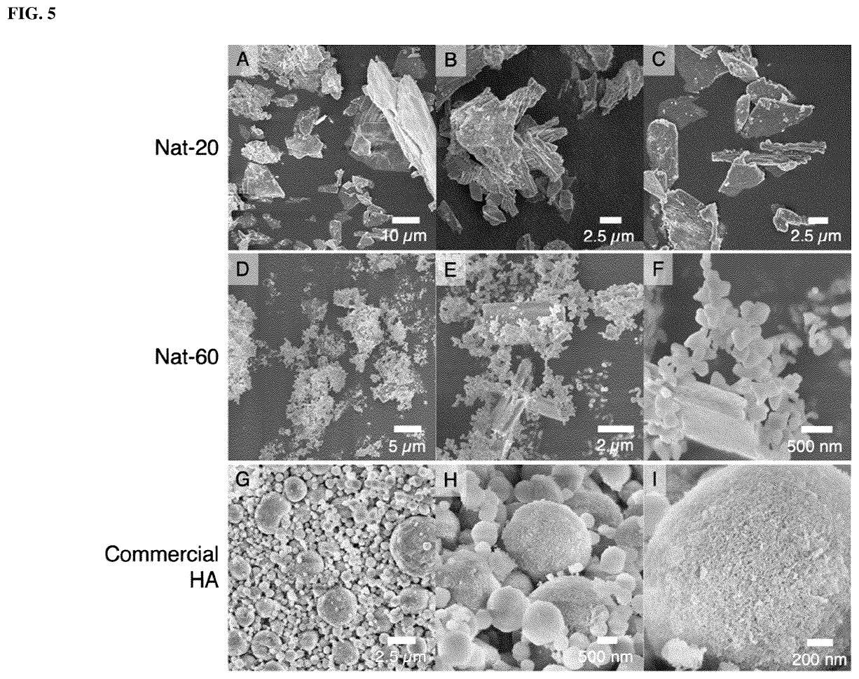

The SEM images of synthesized samples (FIGS. 4 and 5) provide morphological details. All samples in the pH range of 8-11 show primary particle sizes in the approximate range of 20-200 nm, which is quite consistent with comparable literature reports. Nanoparticles appear to aggregate into a variety of structures, including both shard-like and sphere-like agglomerates. The level of agglomeration is high, which is expected as dispersants were not employed. Separately, lower reaction temperatures may contribute more amorphous structures, which may explain the larger degree of plate-like geometries in the samples synthesized at 20° C. in comparison to 60° C. Though this trend does not manifest for CaP-8-60, this may be partially explained by the relatively low synthesis pH of this sample. Otherwise, variations in synthesis pH from 8-11 do not appear to significantly impact the morphology.

The aforementioned samples primarily encompass HA/CDHA and/or R-TCP-based structures, as elucidated via FTIR. In contrast, natural pH samples (FIGS. 1, 3) are composed mainly of γ-CPP. Similar to HA and TCP-based structures, γ-CPP may form a large variety of micro- and nanostructures, including nanoscale particles and rods as well as agglomerates that tend towards plate-like geometries. Both CaP-Nat-20 and CaP-Nat-60 demonstrate these characteristics, matching well with previous accounts of brushite calcined to achieve γ-CPP. One difference between these samples is the higher tendency of CaP-Nat-60 to form nanoparticles. This may indicate that CaPs with lower Ca/P ratios may also exhibit the above-mentioned trend, where less amorphous structures are promoted through higher synthesis temperatures. However, this factor may also be related to the chosen resting time of 24 h. This duration is reported to produce γ-CPP morphologies on the cusp between plate-like and particle-like, while extending the resting time by several days results in plate-like geometries dominating. Across all synthesized samples, there may be a tendency towards greater aggregation and larger feature sizes at lower synthesis temperatures—however, there are some outliers to this trend (e.g., CaP-8-60).

### Solar Reflectance Performance

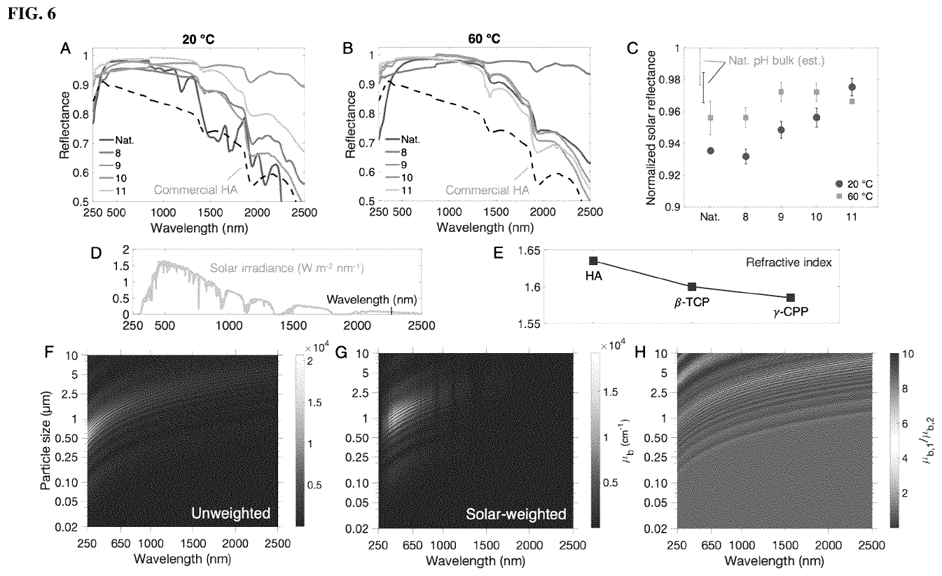

Finally, spectral reflectance from 250-2500 nm is presented for all synthesized CaPs, as shown in FIG. 6a-b. Most spectra present similar wavelength-dependent profiles, with reflectance typically peaking within the visible region and declining towards infrared (IR) wavelengths. A reflectance of well over 0.9 is provided by nearly all synthesized CaPs throughout the peak solar wavelength region, which bodes well for solar-reflective applications.

The resemblance in spectral reflectance between samples may be due to similarity in two factors: the refractive index and the feature sizes of the synthesized powders. There are only slight differences in the refractive indices of HA, TCP, and CPP, as shown in FIG. 6d.—all have an index in the visible region close to 1.6. A higher refractive index may be desirable for high-reflectance applications, but the differences between CaPs synthesized here are minimal.

Together, the refractive index and particle size of a medium determine the scattering properties of a diffuse-reflecting material. The backscattering efficiency is proportional to its reflectance and can be calculated as described elsewhere in the literature. By assuming spherical particle sizes, a refractive index of 1.6, and a dispersion of 5% CaP particles in an air medium, the backscattering efficiency (μb) can be calculated for a given particle size and incident wavelength. The extinction coefficient is set at a conservative value of 10⁻⁶ based on similarities with other low index reflecting materials. It is also noted that, while non-spherical morphologies may require different treatment for more accurate values of μb, the strategy described herein is sufficient to illustrate the general trend between μb and feature sizes.

As shown in FIG. 6f-g, for a refractive index of 1.6, spherical particle sizes around 1 μm may be suitable for reflecting incident light at peak solar wavelengths. While smaller particle sizes can offer scattering coefficients of higher magnitude (FIG. 6f), weighting with respect to the solar spectrum (FIG. 6d) may provide the advantage of highlighting the most critical solar wavelengths. The result of this weighting process (FIG. 6g may indicate that the particle size range of 0.8-2 μm is most effective for scattering solar incidence. Nearly all of the synthesized CaPs have feature sizes which fall within this range, which is identified as a factor in their high normalized solar reflectance values.

Furthermore, while a monodisperse powder is highly effective at scattering a particular wavelength of light, solar radiation is spread across a wide range of wavelengths. When ultra-high solar reflectance applications are desired, a polydisperse powder is better suited to offer a higher overall reflectance due to the broad emission spectrum of the Sun. The synthesized CaPs may offer such a polydisperse powder: while these powders possess nanoscale primary particle sizes, they may aggregate into microscale superstructures (e.g., shards, plates, and spherules, as seen in SEM images). This hierarchical distribution of feature sizes is far better suited to reflect broadband solar radiation and may contribute greatly to the high normalized reflectance values of the samples (FIG. 6c). The commercial HA provides a good basis of comparison: its spectral reflectance is highest at low solar wavelengths but far diminished at longer wavelengths. This may be due to its relatively small and less widely distributed particle sizes, which tend around 500 nm, and have far less variation than the synthesized CaPs (FIG. 5g-i). Though this particle size offers amongst the highest backscattering coefficient values seen in FIG. 6f, weighting with respect to the solar spectrum reveals that this factor may be less impactful for total solar reflectance. In addition, the commercial HA particles themselves may be composed of extremely small grains—too small to effectively scatter solar radiation. Even with a relatively higher refractive index, its unoptimized particle sizes and lack of a useful hierarchical structure may prevent it from realizing ultrahigh solar reflectance. Together, these attributes contribute to its relatively low normalized solar reflectance value of 0.85-about 10% lower than most synthesized CaPs, even with its higher refractive index.

Even with the high spectral reflectance performance of all CaP samples, different samples do exhibit some differences. Samples at higher pH mostly show higher normalized reflectance values. This may be attributed partially to the greater proportion of stoichiometric HA expected from a higher synthesis pH, which offers a minor increase in refractive index that will raise the scattering efficiency for a given particle size. In addition, for a given pH, the raised synthesis temperature of 60° C. may outperform 20° C. samples in terms of normalized reflectance. This is attributed to raised temperatures supporting greater Ca/P ratios, which can also offer an increase in the average refractive index of the sample. The quantitative impact of this is seen in FIG. 6h—for a given particle size and wavelength, the refractive index of HA (about 1.64) results in pb values about 2-4 times higher than those of γ-CPP, with its lower refractive index of about 1.59. Thus, while the refractive indices between samples of different CaP composition may change only slightly, this can contribute to differing solar reflectance values.

The reaction temperature-reflectance trend may also be supported by the variation in morphology between low and high temperature synthesis. As mentioned previously, lower reaction temperatures may promote larger amorphous structures, as seen within the SEM images. Structures in the larger microscale may be beneficial for scattering IR solar wavelengths (as shown in FIG. 6g), but too high of a proportion at this feature size may be detrimental to reflectance at peak solar wavelengths. CaP-10-20 and CaP-8-60 exemplify this characteristic: both present somewhat larger shard-like geometries in their SEM images as compared to other samples. As a consequence, their IR reflectance is significantly higher, but their reflectance at peak solar wavelengths drops, lowering their normalized reflectance values.

Samples with natural pH show very interesting reflectance results. As they are composed of γ-CPP, they offer the lowest refractive index of the identified constituents. Yet, their normalized solar reflectance values as-measured are close to those of the CaP-8 samples. Furthermore, when adjusting for the slight difference in sample preparation for the reflectance characterization (as described in 2.3), the CaP-Nat samples are estimated to present amongst the highest reflectance values. A contributor to this behavior may be the morphology of the CaP-Nat samples, which matches greatly with the requirements of scattering solar radiation at a refractive index around 1.6. Firstly, CaP-Nat samples show a broad range of feature sizes, ranging from as small as several hundred nanometers (e.g., FIG. 5f) to low micron sizes (e.g., FIG. 5a-e, c). These factors supply a broad distribution of feature sizes that coincides nearly exactly with the highest values of μb in FIG. 6g. Their shard-like aggregate structures also show quite high aspect ratios when compared with pH-controlled CaPs—an attribute that has been shown to positively impact total solar reflectance in a similar manner as widened particle size distributions. Though many other CaPs present hierarchical morphology distributions with similar feature sizes, this may be attributed to the somewhat stronger performance of the CaP-Nat samples to morphologies that are better balanced for solar radiation, as well as higher feature aspect ratios. In addition, the higher reflectance of the CaP-Nat-60 as compared to the CaP-Nat-20 sample may be attributed to its higher proportion of nanoscale particles, which provides a more diverse range of morphological scales.

### Example 3: Alternative Ca/P Ratio

Described herein are solar-reflective calcium phosphates synthesized using a wet chemical precipitation method and characterized. The synthesis procedure described herein employs a Ca/P ratio of 0.72. Characterization results for Ca/P ratios of 0.72 and 1.0 are compared to establish if significant differences exist in mass yield or solar-reflective properties. To achieve the ratio of 1.0, the input mass of the calcium source was increased accordingly from the original input mass.

The same materials and methods described herein are employed here. Five unique trials with each Ca/P ratio were performed to establish repeatability.

TABLE 1
Synthesis experiment data

| Test | Date of synthesis | | Mass | Measured |
|---|---|---|---|---|
| no. | (start) | Ca/P ratio | yield (g) | reflectance |
| 1 | Apr. 1, 2024 | 0.72 | 12.34 | 91.0% |
| 2 | Apr. 1, 2024 | 0.72 | 12.24 | 91.7% |
| 3 | Apr. 2, 2024 | 0.72 | 12.23 | 92.7% |
| 4 | Apr. 2, 2024 | 0.72 | 12.41 | 90.7% |
| 5 | Apr. 2, 2024 | 0.72 | 12.08 | 91.9% |
| 6 | May 22, 2024 | 1.00 | 12.93 | 91.9% |
| 7 | May 23, 2024 | 1.00 | 13.66 | 92.1% |
| 8 | May 28, 2024 | 1.00 | 13.61 | 91.5% |
| 9 | May 29, 2024 | 1.00 | 13.70 | 91.5% |
| 10 | May 29, 2024 | 1.00 | 13.42 | 91.8% |

TABLE 2
Overall results for each Ca/P ratio test group

| | | | | Standard |
|---|---|---|---|---|
| Ca/P | Average mass | Standard deviation | Average | deviation |
| ratio | yield (g) | mass yield (g) | reflectance | reflectance |
| 0.72 | 12.27 | 0.13 | 91.6% | 0.8% |
| 1.00 | 13.47 | 0.32 | 91.8% | 0.3% |

As can be seen by the results in Tables 1 and 2, a synthesis with an increased Ca/P ratio of 1.0 has been successfully demonstrated. This has resulted in a greater mass yield with no significant negative impact on the measured solar reflectance. These results are repeatable over multiple experiments.

### Example 4: Potential Applications

Many of the synthesis parameters described herein lie on the cusp of mechanism transitions. For example, the calcination temperature of 700° C. may both β-TCP and CDHA to manifest, whereas a slightly higher temperature would promote only BCP (β-TCP and stoichiometric HA). The prescribed resting time of 24 h for CaP-Nat samples produces a mixture of both particle-like and plate-like geometries, while longer resting times may provide a mainly plate-like morphology in the resulting γ-CPP. Also, the starting pH of 8 lies on the line between CDHA and dicalcium phosphate precipitation, and the resulting composition appears to be sensitively dependent on synthesis temperature at this pH. For certain scenarios—such as medical applications where a specific, homogeneous CaP composition and morphology is demanded—these intermediate synthesis parameters may prove problematic. However, such intermediate synthesis parameters may be valuable in applications where a broad range of features are desirable, such as offering multifunctional medical composites with varied properties.

The processes described herein may be particularly suitable for high solar reflectance applications, as it produces dispersed morphologies and refractive indices that can address the broad range of wavelengths in the solar emission spectrum. The advantage of properly sized calcium phosphates (as have been demonstrated herein) is pronounced with respect to industry-standard white pigments such as titanium dioxide (TiO2). While TiO2 exhibits high opacity due to its large refractive index, its low optical bandgap with respect to calcium phosphates results in UV absorbance, hindering its ability to act as a near-ideal solar reflective pigment. For this reason, solar-reflective paints with alternative pigments including calcium phosphates, calcium carbonate (CaCO3), and barium sulfate (BaSO4) have demonstrated success in radiative cooling paints, often outperforming TiO2-based comparisons. Thus, proper sizing of these and other similar materials (which have relatively similar refractive indices and larger bandgaps than TiO2) may be leveraged to achieve similarly strong results for broadband solar reflectance.

In addition, for passive radiative cooling applications, maximizing both broadband solar reflectance and mid-IR emittance (especially in the atmospheric transparency region of 8-13 μm) may be essential for optimizing cooling performance. The materials and processes described herein characterize only the solar reflectance portion of this requirement. However, for actual applications, particle-based materials, such as the calcium phosphates exemplified herein, are most commonly and most easily dispersed within organic polymer matrices to create paint-like approaches for passive radiative cooling. In these instances, the polymer coatings themselves provide substantial mid-IR emittance due to the phonon modes of typical monomer constituents. For this reason, high mid-IR emittance of a passive radiative cooling paint incorporating the calcium phosphates described herein may be easily attained through typical paint matrices, without having to rely on the emittance of the calcium phosphates themselves.

### REFERENCES

[1]R. Z. LeGeros, Calcium Phosphate-Based Osteoinductive Materials, Chemical Reviews 108 (11) (2008) 4742-4753, publisher: American Chemical Society. doi:10.1021/cr800427g.
URL https://doi.org/10.1021/cr800427g
[2]H. Yuan, Z. Yang, Y. Li, X. Zhang, J. D. De Bruijn, K. De Groot, Osteoinduction by calcium phosphate biomaterials, Journal of Ma-terials Science: Materials in Medicine 9 (12) (1998) 723-726. doi:10.1023/A:1008950902047.
URL https://doi.org/10.1023/A:1008950902047
[3]M.-P. Ginebra, C. Canal, M. Espanol, D. Pastorino, E. B. Montufar, Calcium phosphate cements as drug delivery materials, Advanced Drug Delivery Reviews 64 (12) (2012) 1090-1110. doi:10.1016/j.addr.2012.01.008.
URL https://www.sciencedirect.com/science/article/pii/S0169409X12000117
[4]P. Wang, C. Li, H. Gong, X. Jiang, H. Wang, K. Li, Effects of synthesis conditions on the morphology of hydroxyapatite nanoparticles produced by wet chemical process, Powder Technology 203 (2) (2010) 315-321. doi:10.1016/j.powtec.2010.05.023.
URL https://www.sciencedirect.com/science/article/pii/S003259101000272X
[5]Y. Liu, D. Hou, G. Wang, A simple wet chemical synthesis and characterization of hydroxyapatite nanorods, Materials Chemistry and Physics 86 (1) (2004) 69-73. doi:10.1016/j.matchemphys.2004.02.009.
URL https://www.sciencedirect.com/science/article/pii/S0254058404001002
[6]S. Kannan, A. F. Lemos, J. M. F. Ferreira, Synthesis and Mechanical Performance of Biological-like Hydroxyapatites, Chemistry of Materials 18 (8) (2006) 2181-2186, publisher: American Chemical Society. doi:10.1021/cm052567q.
URL https://doi.org/10.1021/cm052567q
[7]I. Lodoso-Torrecilla, J. J. J. P. van den Beucken, J. A. Jansen, Calcium phosphate cements: Optimization toward biodegradability, Acta Biomaterialia 119 (2021) 1-12. doi:10.1016/j.actbio.2020.10.013.
URL https://www.sciencedirect.com/science/article/pii/S1742706120306024
[8]J. Zhang, W. Liu, V. Schnitzler, F. Tancret, J.-M. Bouler, Calcium phosphate cements for bone substitution: Chemistry, handling and mechanical properties, Acta Biomaterialia 10 (3) (2014) 1035-1049. doi:10.1016/j.actbio.2013.11.001.
URL https://www.sciencedirect.com/science/article/pii/S1742706113005576
[9]A. J. Ambard, L. Mueninghoff, Calcium Phosphate Cement: Review of Mechanical and Biological Properties, Journal of Prosthodontics 15 (5) (2006) 321-328,_eprint: https://onlinelibrary.wiley.com/doi/pdf/10.1111/j.1532-849X.2006.00129.x. doi:10.1111/j.1532-849X.2006.00129.x.
URL https://onlinelibrary.wiley.com/doi/abs/10.1111/j.1532-849X.2006.00129.x
[10]J. R. Cunha, C. Schott, R. D. van der Weijden, L. H. Leal, G. Zeeman, C. Buisman, Calcium phosphate granules recovered from black water treatment: A sustainable substitute for mined phosphorus in soil fertilization, Resources, Conservation and Recycling 158 (2020) 104791. doi:10.1016/j.resconrec.2020.104791.
URL https://www.sciencedirect.com/science/article/pii/S0921344920301129
[11]F. Gaiotti, M. Lucchetta, G. Rodegher, D. Lorenzoni, E. Longo, E. Boselli, S. Cesco, N. Belfiore, L. Lovat, J. M. Delgado-López, F. J. Carmona, A. Guagliardi, N. Masciocchi, Y. Pii, Urea-Doped Calcium Phosphate Nanoparticles as Sustainable Nitrogen Nanofertilizers for Viticulture: Implications on Yield and Quality of Pinot Gris Grapevines, Agronomy 11 (6) (2021) 1026, number: 6 Publisher: Multidisciplinary Digital Publishing Institute. doi:10.3390/agronomy11061026. URL https://www.mdpi.com/2073-4395/11/6/1026
[12]F. J. Carmona, G. Dal Sasso, G. B. Ramírez-Rodríguez, Y. Pii, J. M. Delgado-López, A. Guagliardi, N. Masciocchi, Urea-functionalized amorphous calcium phosphate nanofertilizers: optimizing the synthetic strategy towards environmental sustainability and manufacturing costs, Scientific Reports 11 (1) (2021) 3419, publisher: Nature Publishing Group. doi:10.1038/s41598-021-83048-9. URL https://www.nature.com/articles/s41598-021-83048-9
[13]S. Feng, Y. Zhou, C. Liu, T. Zhang, X. Bu, Y. Huang, M. He, Skeleton-inspired optical-selective cellulose-based bio-film as passive radiative cooler and the energy-saving performance evaluation, Chemical Engineering Journal 452 (2023) 139377. doi:10.1016/j.cej.2022.139377.
URL https://www.sciencedirect.com/science/article/pii/S1385894722048562
[14]J. Lv, Z. Chen, X. Li, Calcium Phosphate Paints for Full-Daytime Subambient Radiative Cooling, ACS Applied Energy Materials 5 (4) (2022) 4117-4124, publisher: American Chemical Society. doi:10.1021/acsaem.1c03457. URL https://doi.org/10.1021/acsaem.1c03457
[15]Y. Tian, X. Liu, Z. Wang, J. Li, Y. Mu, S. Zhou, F. Chen, M. L. Minus, G.Xiao, Y. Zheng, Subambient daytime cooling enabled by hierarchically architected all-inorganic metapaper with enhanced thermal dissipation, Nano Energy 96 (2022) 107085. doi:10.1016/j.nanoen.2022.107085.
URL https://www.sciencedirect.com/science/article/pii/S2211285522001665
[16]A. Caratenuto, K. Leach, Y. Liu, Y. Zheng, Nanofibrous Biomaterial-Based Passive Cooling Paint Structurally Linked by Alkane-Oleate Interactions, ACS Applied Materials & Interfaces 16 (10) (2024) 12717-12730, publisher: American Chemical Society. doi:10.1021/acsami.4c01383. URL https://doi.org/10.1021/acsami.4c01383
[17]M. Bin Mobarak, M. S. Hossain, Z. Yeasmin, M. Mahmud, M. M. Rahman, S. Sultana, S. M. Masum, S. Ahmed, Probing the photocatalytic competency of hydroxyapatite synthesized by solid state and wet chemical precipitation method, Journal of Molecular Structure 1252 (2022) 132142. doi:10.1016/j.molstruc.2021.132142.
URL https://www.sciencedirect.com/science/article/pii/S022286021022626
[18]B. Le Gars Santoni, L. Niggli, G. A. Sblendorio, D. T. L. Alexander, C. Stähli, P. Bowen, N. Döbelin, M. Bohner, Chemically pure β-tricalcium phosphate powders: Evidence of two crystal structures, Journal of the European Ceramic Society 41 (2) (2021) 1683-1694. doi:10.1016/j.jeurceramsoc.2020.09.055.
URL https://www.sciencedirect.com/science/article/pii/S0955221920307822
[19]S. Raynaud, E. Champion, D. Bernache-Assollant, P. Thomas, Calcium phosphate apatites with variable Ca/P atomic ratio I. Synthesis, characterisation and thermal stability of powders, Biomaterials 23 (4) (2002) 1065-1072. doi:10.1016/S0142-9612(01)00218-6.
URL https://www.sciencedirect.com/science/article/pii/S0142961201002186
[20]E. I. Dorozhkina, S. V. Dorozhkin, Mechanism of the Solid-State Transformation of a Calcium-Deficient Hydroxyapatite (CDHA) into Biphasic Calcium Phosphate (BCP) at Elevated Temperatures, Chemistry of Materials 14 (10) (2002) 4267-4272. doi:10.1021/cm0203060.
URL https://pubs.acs.org/doi/10.1021/cm0203060
[21]M. Asadi-Eydivand, M. Solati-Hashjin, A. Farzadi, N. A. A. Osman, Artificial neural network approach to estimate the composition of chemically synthesized biphasic calcium phosphate powders, Ceramics International 40 (8, Part A) (2014) 12439-12448. doi:10.1016/j.ceramint.2014.04.095.
URL https://www.sciencedirect.com/science/article/pii/S0272884214006464
[22]L. Berzina-Cimdina, N. Borodajenko, Research of Calcium Phosphates Using Fourier Transform Infrared Spectroscopy, in: T. Theophanides (Ed.), Infrared Spectroscopy—Materials Science, Engineering and Technology, InTech, 2012. doi:10.5772/36942.
URL http://www.intechopen.com/books/infrared-spectroscopy-materials-science-engineeri
[23]A. Siddharthan, S. K. Seshadri, T. S. S. Kumar, Microwave accelerated synthesis of nanosized calcium deficient hydroxyapatite, Journal of Materials Science: Materials in Medicine 15 (12) (2004) 1279-1284. doi:10.1007/s10856-004-5735-3.
URL https://doi.org/10.1007/s10856-004-5735-3
[24]A. Destainville, E. Champion, D. Bernache-Assollant, E. Laborde, Synthesis, characterization and thermal behavior of apatitic tricalcium phosphate, Materials Chemistry and Physics 80 (1) (2003) 269-277. doi:10.1016/S0254-0584(02)00466-2.
URL https://www.sciencedirect.com/science/article/pii/S0254058402004662
[25]S.-C. Liou, S.-Y. Chen, Transformation mechanism of different chemically precipitated apatitic precursors into β-tricalcium phosphate upon calcination, Biomaterials 23 (23) (2002) 4541-4547. doi:10.1016/S0142-9612(02)00198-9.
URL https://www.sciencedirect.com/science/article/pii/S0142961202001989
[26]S. Dasgupta, A. Bandyopadhyay, S. Bose, Reverse micelle-mediated synthesis of calcium phosphate nanocarriers for controlled release of bovine serum albumin, Acta Biomaterialia 5 (8) (2009) 3112-3121. doi:10.1016/j.actbio.2009.04.031.
URL https://www.sciencedirect.com/science/article/pii/S1742706109001950
[27]B. Viswanath, V. V. Shastry, U. Ramamurty, N. Ravishankar, Effect of calcium deficiency on the mechanical properties of hydroxyapatite crystals, Acta Materialia 58 (14) (2010) 4841-4848. doi:10.1016/j.actamat.2010.05.019.
URL https://www.sciencedirect.com/science/article/pii/S1359645410002946
[28]R. Chanfrau, A. Tolaba, T. Pelizaro, Y. Veranes-Pantoja, G. Carlos, Evaluation of the Behavior in Water of a Biomaterial Composed of Different Phases of Calcium Phosphate, Letters in Applied NanoBioScience, 9(2020)1513-1520. doi:10.33263/LIANBS94.15131520.
[29]E. Salimi, J. Javadpour, Synthesis and Characterization of Nanoporous Monetite Which Can Be Applicable for Drug Carrier, Journal of Nanomaterials 2012 (November 2012). doi:10.1155/2012/931492.
[30]B. O. Fowler, E. C. Moreno, W. E. Brown, Infra-red spectra of hydroxyapatite, octacalcium phosphate and pyrolysed octacalcium phosphate, Archives of Oral Biology 11 (5) (1966) 477-492. doi:10.1016/0003-9969(66)90154-3.
URL https://www.sciencedirect.com/science/article/pii/0003996966901543
[31]D. Griesiute, J. Gaidukevic, A. Zarkov, A. Kareiva, Synthesis of β-Ca2P2O7 as an Adsorbent for the Removal of Heavy Metals from Water, Sustainability 13 (14) (2021) 7859, number: 14 Publisher: Multidisciplinary Digital Publishing Institute. doi:10.3390/su13147859.
URL https://www.mdpi.com/2071-1050/13/14/7859
[32]J. P. Maity, T.-J. Lin, H. P.-H. Cheng, C.-Y. Chen, A. S. Reddy, S. B. Atla, Y.-F. Chang, H.-R. Chen, C.-C. Chen, Synthesis of Brushite Particles in Reverse Microemulsions of the Biosurfactant Surfactin, International Journal of Molecular Sciences 12 (6) (2011) 3821-3830. doi:10.3390/ijms12063821.
URL https://www.ncbi.nlm.nih.gov/pmc/articles/PMC3131593/
[33]D. Lee, P. N. Kumta, Chemical synthesis and stabilization of magnesium substituted brushite, Materials Science and Engineering: C 30 (7)(2010) 934-943. doi:10.1016/j.msec.2010.04.007.
URL https://www.sciencedirect.com/science/article/pii/S0928493110000937
[34]D. Griesiute, E. Garskaite, A. Antuzevics, V. Klimavicius, V. Balevicius, A. Zarkov, A. Katelnikovas, D. Sandberg, A. Kareiva, Synthesis, structural and luminescent properties of Mn-doped calcium pyrophosphate (Ca2P2O7) polymorphs, Scientific Reports 12 (1) (2022) 7116, publisher: Nature Publishing Group. doi:10.1038/s41598-022-11337-y.
URL https://www.nature.com/articles/s41598-022-11337-y
[35]P. Gras, C. Rey, O. Marsan, S. Sarda, C. Combes, Synthesis and Characterisation of Hydrated Calcium Pyrophosphate Phases of Biological Interest, European Journal of Inorganic Chemistry 2013 (34) (2013) 5886-5895, _eprint: https://onlinelibrary.wiley.com/doi/pdf/10.1002/ejic.201300955. doi:10.1002/ejic.201300955.
URL https://onlinelibrary.wiley.com/doi/abs/10.1002/ejic.201300955
[36]Z. Zyman, M. Epple, A. Goncharenko, D. Rokhmistrov, O. Prymak, K. Loza, Peculiarities in thermal evolution of precipitated amorphous calcium phosphates with an initial Ca/P ratio of 1:1, Journal of Materials Science: Materials in Medicine 28 (3) (2017) 52. doi:10.1007/s10856-016-5820-4.
URL https://doi.org/10.1007/s10856-016-5820-4
[37]B. Le Gars Santoni, L. Niggli, S. Dolder, O. Loeffel, G. A. Sblendorio, R. Heuberger, Y. Maazouz, C. Stähli, N. Döbelin, P. Bowen, W. Hofstetter, M. Bohner, Effect of minor amounts of β-calcium pyrophosphate and hydroxyapatite on the physico-chemical properties and osteoclastic resorption of β-tricalcium phosphate cylinders, Bioactive Materials 10 (2022) 222-235. doi:10.1016/j.bioactmat.2021.09.003.
URL https://www.sciencedirect.com/science/article/pii/S2452199X21004138
[38]A. Yelten-Yilmaz, S. Yilmaz, Wet chemical precipitation synthesis of hydroxyapatite (HA) powders, Ceramics International 44 (8) (2018) 9703-9710. doi:10.1016/j.ceramint.2018.02.201.
URL https://www.sciencedirect.com/science/article/pii/S0272884218305169
[39]E. Kramer, J. Podurgiel, M. Wei, Control of hydroxyapatite nanoparticle morphology using wet synthesis techniques: Reactant addition rate effects, Materials Letters 131 (2014) 145-147. doi:10.1016/j.matlet.2014.05.105.
URL https://www.sciencedirect.com/science/article/pii/S0167577X14009173
[40]W. Michał, D. Ewa, C. Tomasz, Lecithin-based wet chemical precipitation of hydroxyapatite nanoparticles, Colloid and Polymer Science 293 (5) (2015) 1561-1568. doi:10.1007/s00396-015-3557-0.
URL https://doi.org/10.1007/s00396-015-3557-0
[41]J. Vilchis-Granados, F. Granados-Correa, C. E. Barrera-Díaz, Surface fractal dimensions and textural properties of mesoporous alkaline-earth hydroxyapatites, Applied Surface Science 279 (2013) 97-102.doi:10.1016/j.apsusc.2013.04.042.
URL https://www.sciencedirect.com/science/article/pii/S0169433213007332
[42]L. Vecbiskena, Z. Irbe, K. Salma-Ancane, L. Berzina-Cimdina, Characterization of Bone Graft Substitutes Prepared by Transformation of β-Tricalcium Phosphate to Hydroxyapatite in Aqueous Environment, European cells & materials 20 (2010) 263.
[43] M. S. Djošić, V. B. Miškovid-Stanković, S. Milonjić, Z. M. Kačarević-Popović, N. Bibić, J. Stojanović, Electrochemical synthesis and characterization of hydroxyapatite powders,
Materials Chemistry and Physics111 (1) (2008) 137-142. doi:10.1016/j.matchemphys.2008.03.045.
URL https://www.sciencedirect.com/science/article/pii/S0254058408001703
[44]T. V. Safronova, T. B. Shatalova, S. A. Tikhonova, Y. Y. Filippov, V. K. Krut'ko, O. N. Musskaya, N. E. Kononenko, Synthesis of Calcium Pyrophosphate Powders from Phosphoric Acid and Calcium Carbonate, Inorganic Materials: Applied Research 12 (4) (2021) 986-992. doi:10.1134/S2075113321040353.
URL https://doi.org/10.1134/S2075113321040353
[45]T. Safronova, A. Kiselev, I. Selezneva, T. Shatalova, Y. Lukina, Y. Filippov, O. Toshev, S. Tikhonova, O. Antonova, A. Knotko, Bioceramics Based on β-Calcium Pyrophosphate, Materials 15 (9) (2022) 3105, number: 9 Publisher: Multidisciplinary Digital Publishing Institute. doi:10.3390/ma15093105.
URL https://www.mdpi.com/1996-1944/15/9/3105
[46]T. Safronova, V. Putlayev, K. Bessonov, V. Ivanov, Ceramics based on calcium pyrophosphate nanopowders, Processing and Application of Ceramics 7 (1) (2013) 9-14. doi:10.2298/PAC1301009S. URL https://doiserbia.nb.rs/Article.aspx?ID=1820-61311301009S
[47]M. Furko, Z. E. Horváth, J. Mihily, K. Balázsi, C. Balazsi, Comparison of the Morphological and Structural Characteristic of Bioresorbable and Biocompatible Hydroxyapatite-Loaded Biopolymer Composites, Nanomaterials 11 (12) (2021) 3194, number: 12 Publisher: Multidisciplinary Digital Publishing Institute. doi:10.3390/nano11123194. URL https://www.mdpi.com/2079-4991/11/12/3194
[48]D. Holzmann, D. Holzinger, G. Hesser, T. Schmidt, G. Knör, Hydroxyapatite Nanoparticles as novel Low-Refractive Index Additives for the Long-Term UV-Photoprotection of Transparent Composite Materials., Journal of Materials Chemistry 19 (2009) 8102. doi:10.1039/B912116A.
[49]A. S. Posner, S. R. Stephenson, Crystallographic investigation of tricalcium phosphate hydrate, Journal of Dental Research 31 (3) (1952) 371-382.
doi:10.1177/00220345520310031501.
[50] PubChem, Calcium pyrophosphate.
URL https://pubchem.ncbi.nlm.nih.gov/compound/24632
[51]J. Peoples, X. Li, Y. Lv, J. Qiu, Z. Huang, X. Ruan, A strategy of hierarchical particle sizes in nanoparticle composite for enhancing solar reflection, International Journal of Heat and Mass Transfer 131 (2019) 487-494. doi:10.1016/j.ijheatmasstransfer.2018.11.059.
URL https://www.sciencedirect.com/science/article/pii/S0017931018334665
[52] X. Li, J. Peoples, P. Yao, X. Ruan, Ultrawhite BaSO4 Paints and Films for Remarkable Daytime Subambient Radiative Cooling, ACS Applied Materials & Interfaces 13 (18) (2021) 21733-21739. doi:10.1021/acsami.1c02368.
URL https://doi.org/10.1021/acsami.1c02368
[53] X. Li, J. Peoples, Z. Huang, Z. Zhao, J. Qiu, X. Ruan, Full Daytime Sub-ambient Radiative Cooling in Commercial-like Paints with High Figure of Merit, Cell Reports Physical Science 1 (10), publisher: Elsevier (October 2020). doi:10.1016/j.xcrp.2020.100221.
URL https://www.cell.com/cell-reports-physical-science/abstract/S2666-3864(20)30236-8
[54]Z. Tong, J. Peoples, X. Li, X. Yang, H. Bao, X. Ruan, Electronic and phononic origins of BaSO4 as an ultra-efficient radiative cooling paint pigment, Materials Today Physics 24 (2022) 100658. doi:10.1016/j.mtphys.2022.100658.
URL https://www.sciencedirect.com/science/article/pii/S2542529322000566
[55]A. Caratenuto, S. Leung, N. LeCompte, Y. Zheng, Size-dispersed calcium phosphate-based paints for sustainable, durable cool roof applications, Energies 17 (16) (2024) 4178.
[56]J. Peoples, X. Ruan, Chapter 18—Radiative cooling paints, in: M. P. Mengüç, M. Francoeur (Eds.), Light, Plasmonics and Particles, Nanophotonics, Elsevier, 2023, pp. 393-419. doi:10.1016/B978-0-323-99901-4.00029-9.
URL https://www.sciencedirect.com/science/article/pii/B9780323999014000299
[57]F. P. Incropera, F. P. Incropera (Eds.), Fundamentals of Heat and Mass Transfer, 6th Edition, John Wiley, Hoboken, NJ, 2007.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A material, comprising one or more calcium phosphates, wherein the material is a polydisperse powder comprising a plurality of particles of differing sizes.

2. The material of claim 1, wherein the one or more calcium phosphates are selected from calcium pyrophosphate (CPP), biphasic calcium phosphate (BCP), hydroxyapatite (HA), calcium-deficient HA (CDHA), tricalcium phosphate (TCP), and a combination of any of them.

3. The material of claim 2, wherein the one or more calcium phosphates is calcium pyrophosphate.

4.-6. (canceled)

7. The material of claim 2, wherein the one or more calcium phosphates is tricalcium phosphate (TCP).

8.-9. (canceled)

10. The material of claim 1, wherein the one or more calcium phosphates comprise hydroxyapatite and calcium-deficient hydroxyapatite.

11. The material of claim 1, wherein the plurality of particles has a distribution of particle sizes of about 1 nm to about 400 nm.

12. (canceled)

13. The material of claim 1, wherein the plurality of particles form a plurality of aggregates.

14. The material of claim 13, wherein the plurality of aggregates comprises amorphous aggregates.

15. The material of claim 13, wherein the plurality of aggregates comprises microstructures or nanostructures, or both.

16. (canceled)

17. The material of claim 15, wherein the plurality of aggregates comprises one or more morphologies selected form shard-like, plate-like, particles, rods, spherules, and a combination of any of them.

18. (canceled)

19. The material of claim 13, wherein the plurality of aggregates has a distribution of aggregate sizes of about 200 nm to about 10 μm.

20. (canceled)

21. The material of claim 1, wherein the material has a refractive index of about 1.55 to about 1.68.

22.-24. (canceled)

25. The material of claim 1, wherein the material has normalized spectral reflectance values of about 0.85 to about 0.98 in the solar radiation spectrum (250-2500 nm).

26.-37. (canceled)

38. An additive for a passive radiative cooling paint or coating, comprising the material of claim 1.

39. A paint, comprising the material of claim 1.

40. A building material, comprising the material of claim 1.

41. (canceled)

42. A sunscreen, comprising the material of claim 1.

43. A pigment, comprising the material of claim 1.

44. The material of claim 1, wherein the material reflects sunlight.

45. A process of preparing a solar reflective material, comprising:
i) combining a first solution comprising a calcium-containing reagent with a second solution comprising a phosphate-containing reagent at a reaction temperature and a reaction pH, thereby producing a calcium-phosphate mixture comprising calcium-phosphate;
ii) allowing the calcium phosphate to precipitate from the calcium-phosphate mixture; and
iii) calcining the calcium phosphate,
thereby producing the solar reflective material.

46.-88. (canceled)
